(19) United States
(12) Patent Application Publication
(10) Pub. No.: US 2015/0346216 A1
(43) Pub. Date: Dec. 3, 2015

(54) DEVICES AND METHODS FOR THE DIAGNOSIS AND TREATMENT OF WOUNDS USING BIOMARKERS

(71) Applicant: KCI Licensing, Inc., San Antonio, TX (US)

(72) Inventors: Sandra Garcia, San Antonio, TX (US); Amy McNulty, San Antonio, TX (US); Kristine Villarreal, San Antonio, TX (US)

(21) Appl. No.: 14/739,824

(22) Filed: Jun. 15, 2015

Related U.S. Application Data
(63) Continuation of application No. 13/303,921, filed on Nov. 23, 2011.
(60) Provisional application No. 61/416,544, filed on Nov. 23, 2010.

(51) Int. Cl. G01N 33/68; G01N 33/50

(52) U.S. Cl. CPC G01N 33/6872; G01N 33/5091; G01N 2333/503; G01N 2333/908; G01N 2333/966

(57) ABSTRACT

The present invention provides for devices and methods for determining the healing phase of a wound. In some aspects, the present invention provides a wound diagnosis device comprising a surface and at least one agent that is specific to a desired biomarker. In another aspect, the present invention provides a method of determining the phase of wound healing. In still other aspects, the present invention provides methods of determining the phase of wound healing using the disclosed devices.

### CROSS-REFERENCE TO RELATED APPLICATIONS

This application is a continuation of U.S. patent application Ser. No. 13/303,921, filed Nov. 23, 2011, which claims priority to U.S. Provisional Patent Application Ser. No. 61/416,544, filed Nov. 23, 2010, the entire contents of which are expressly incorporated herein by reference.

### BACKGROUND OF THE INVENTION

1. Field of the Invention

The present invention relates generally to the field of biology. More particularly, it relates to devices and methods for the diagnosis and treatment of wounds using biomarkers.

2. Description of the Related Art

Hospital and home care settings treating acute and chronic wounds from various sources (e.g., venous leg, diabetic foot or pressure ulcers) would greatly benefit from a diagnostic tool that allows the healthcare practitioner to instantly determine if a wound is healing or if the healing is delayed. Currently, there are no fast, easy diagnostic devices or methods for determining the phase of wound healing available on the market.

### SUMMARY OF THE INVENTION

The present invention provides for devices and methods for determining whether a wound is within the normal trajectory of healing or to identify the stage where it has arrested, without causing further damage to the patient.

In some aspects, the present invention provides a wound diagnosis device comprising a surface and at least one agent that is specific to a desired biomarker. In some embodiments, the device comprises a solid surface having at least a first agent that is specific to a first biomarker and a least a second agent that is specific to a second biomarker. In some embodiments, the device further comprises at least a third agent that is specific to a third biomarker. In some embodiments, the device comprises 1, 2, 3, 4, 5, 10, 15, 20, 30, 40, 50, 60, 70 80, 90, 100, or more agents that are specific to 1, 2, 3, 4, 5, 10, 15, 20, 30, 40, 50, 60, 70 80, 90, 100, or more biomarkers.

The surface may be any surface to which the desired agents may be attached, including but not limited to a wound dressing or a microarray. In some embodiments, the surface is an adsorbant surface. In some embodiments, the adsorbant surface comprises filter paper or colorimetric paper. In some embodiments, the device is a wound dressing. The wound dressing may be any appropriate type of wound dressing. In some embodiments, the wound dressing comprises a polyurethane film dressing. In some embodiments, the surface is a microarray.

The agent may be any appropriate agent that is specific to a desired biomarker. In some embodiments, the agent may be aptamers, ligands, antibodies, or peptide sequences. In some embodiments, the agents may be a combination of aptamers, ligands, antibodies, and/or peptide sequences in any combination.

The agent may be attached to the surface in any appropriate manner. In some embodiments, the device is coated or bound with agents that bind to the biomarkers. In some embodiments, the agents may be bound chemically (ionic or covalently) or physically to the wound dressing. In some embodiments, the agents are bound by chemical grafting, polymer-binder, bi-functional chemical binding (e.g., silane or pluronic), hydrophilic or electrochemical binding, or plasma coating. In other embodiments, the device may be coated with the agents. In some embodiments, the agents are linked to the solid surface by covalent bonds.

The biomarker may be any secreted biomarker. In some embodiments, the biomarker may be myeloperoxidase (MPO), neutrophil elastase (nElastase), Human Neutrophil Lipolin (HNL), Lactoferrin, Lysozyme, Neutrophil Gelatinase-Associated Lipocalin (NGAL), Human Neutrophil Elastase Anti-Neutrophil Cytoplasmic Antibodies (HNE ANCA's), MMP9, Proteinase 3, Serpin Peptidase Inhibitor Clade B and D, Reactive Oxygen Species (ROS), or Reactive Nitrogen Species (RNS), or any combination thereof. In some embodiments, the biomarker may be basic fibroblast growth factor (FGF-2), Fibroblast Growth Factor-10 (FGF-10), Fibroblast-specific protein 1 (FSP1), prolyl-4-hydroxylase (5B5), Insulin Growth Factor-1 (IGF-1), Tetranectin, Collagen alpha 1, 2, and 3 chains, SERPINA1, or Compliment Components or any combination thereof. In other embodiments, the biomarker may be calgranulin A/B, Cystatin A, 5100 Calcium Binding Proteins, CD163, CD204, CD206, AM-3K, CSF-1R (colony-stimulating factor-1 receptor), a specific marker of macrophages, EMR1 (epidermal growth factor module-containing mucin-like receptor 1), F4/80, pro-collagen, collagen, or fibronectin or any combination thereof. Any of these biomarkers may be used alone or in combination.

In some embodiments, the first biomarker is MPO and the second biomarker is FGF-2. In some embodiments, the first biomarker is nElastase and the second biomarker is FGF-10. In some embodiments, the first biomarker is calgranulin A/B, TNF-α, MPO, or nElastase and the second biomarker is CD204 or CD206. In some embodiments, the first biomarker is pro-collagen or collagen and the second biomarker is fibronectin.

In another aspect, the present invention provides a method of determining the phase of wound healing comprising: a) detecting the amount of a first biomarker and the amount of a second biomarker in a sample from a wound; and b) comparing the amount of the first biomarker and the amount of the second biomarker, wherein the relation between the amount of the first biomarker to the amount of the second biomarker indicates the phase of wound healing.

The sample that is analyzed by the devices and methods disclosed herein may be from any desirable source. In some embodiments, the sample may be from a wound exudate. In some embodiments, the sample is wound fluid. In some embodiments, the sample may be collected from other sources. For example, the biomarkers may be extracted from tissue which is either adhered to dressing material (upon removal of the dressing from the wound) or from non-necrotic tissue removed during debridement of the wound.

In some embodiments, the first biomarker has been secreted from neutrophil cells and the second biomarker has been secreted from fibroblast cells. In some embodiments, if the amount of the first biomarker is greater than the amount of the second biomarker, the inflammatory phase of wound healing is indicated. Conversely, if the amount of the first biomarker is less than the amount of the second biomarker, the proliferative phase of wound healing is indicated.

In some embodiments, the first biomarker is MPO and the second biomarker is FGF-2. In some embodiments, if the amount of MPO is at least twice greater than the amount of FGF-2, then the inflammatory phase of wound healing is indicated. Conversely, if the amount of MPO is half as much or less than the amount of FGF-2, then the proliferative stage of wound healing is indicated.

In some embodiments, the first biomarker is nElastase and the second biomarker is Fibroblast Growth Factor-10 (FGF-10). In some embodiments, the first biomarker has been secreted from M1 macrophage cells and the second biomarker has been secreted from M2 macrophage cells. In some embodiments, a larger amount of the first biomarker secreted from M1 macrophage cells and a smaller amount of the second biomarker secreted from M2 macrophage cells indicates the inflammatory phase of wound healing. Conversely, a smaller amount of the first biomarker secreted from M1 macrophage cells and a larger amount of the second biomarker secreted from M2 macrophage cells indicates the proliferative phase of wound healing. In some embodiments, the first biomarker is calgranulin A/B, TNF-α, MPO, or MMP and the second biomarker is CD204 or CD206. In some embodiments, if the amount of CD204 or CD206 is less than twice the amount of calgranulin A/B, TNF-α, MPO, or MMP, then the wound is in the inflammatory phase of wound healing. Conversely, if the amount of CD204 or CD206 is equal to or greater than twice the amount of calgranulin A/B, TNF-α, MPO, or MMP, then the wound is in the proliferative stage of wound healing.

In some embodiments, the first biomarker is pro-collagen or collagen and the second biomarker is fibronectin. In some embodiments, if the amount of pro-collagen or collagen is greater than the amount of fibronectin, the inflammatory phase of wound healing is indicated. Conversely, if the amount of pro-collagen or collagen is less than the amount of fibronectin, the proliferative phase of wound healing is indicated.

In some aspects, the present invention provides a method of determining the phase of wound healing comprising: a) detecting the amount of a first biomarker and a second biomarker in a sample from a wound comprising contacting the sample with a solid surface comprising a first agent that is specific to the first biomarker and a second agent that is specific to the second biomarker; and b) comparing the amount of the first biomarker and the amount of the second biomarker, wherein the relative amount of the first biomarker to the second biomarker indicates the phase of wound healing.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

The term “therapeutically effective” as used herein refers to an amount of cells and/or therapeutic composition (such as a therapeutic polynucleotide and/or therapeutic polypeptide) that is employed in methods of the present invention to achieve a therapeutic effect, such as wherein at least one symptom of a condition being treated is at least ameliorated.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 Schematic diagram of diagnostic detection device for chronic wounds.

FIG. 2 ELISA analysis of neutrophil- and fibroblast-specific biomarkers.

FIG. 3 Schematic diagram of digital diagnostic device for rapid detection of status of a wound.

### DESCRIPTION OF THE ILLUSTRATIVE EMBODIMENTS

The disclosed devices and methods provide an easy, rapid, bed-side diagnostic of the wound status. This provides clinicians with a decision tree on “what to do next” by providing information to the healing status of the wound.

Here disclosed are devices and methods useful to detect one or more biomarkers from wound fluid, without disrupting the underlying wounded tissue, to accurately diagnose whether a wound is within the normal trajectory of healing or to identify the stage where it has arrested, without causing further damage to the patient. In general, diagnostic tests are presented that (1) detect secreted biomarkers and (2) measure the ratio of these markers in a sample. These diagnostic tests will be indicative of whether a wound is stalled in the inflammatory phase or has progressed into the proliferative phase of wound healing without disrupting the underlying damaged tissue.

### A. WOUND HEALING PHASES

The wound healing process is subdivided into three phases: (1) inflammation, (2) proliferation and (3) remodeling. The time course of the different cells that appear in the wound during the healing process has been characterized extensively. For example, neutrophils are predominant during inflammation whereas fibroblasts are predominant during the proliferative phase. As an initial reaction to a wound, the body mounts an inflammatory response which is characterized by the recruitment of granulocytes, predominantly neutrophils, and cells from the monocyte-macrophage lineage to the site of injury. While neutrophilic inflammation is traditionally regarded as a normal acute response, the continual presence of neutrophils is often associated with delayed wound healing and chronically stalled wounds. In the normal trajectory of wound healing, the proliferative phase, where epithelialization, angiogenesis and provisional matrix formation takes place, is typically initiated after neutrophils have been cleared out by activated macrophages. Therefore, fibroblasts are the predominant cell-type found during the proliferative phase of a healthy, healing wound.

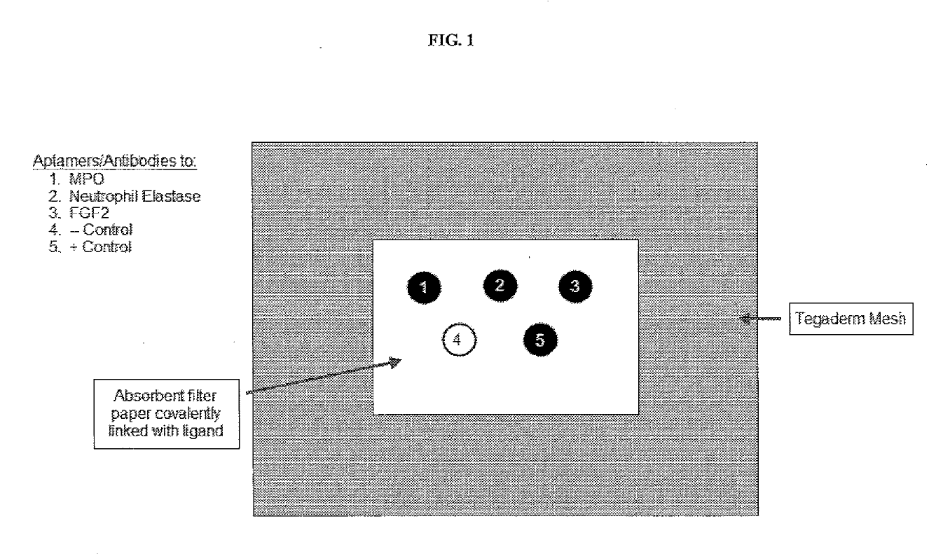

Detection of specific biomarkers secreted by these and other healing phase-specific cell types will allow for accurate determination of wound healing progression as an indirect but accurate representation of the predominant cell type in the wounded tissue. In the methods disclosed herein, unique markers secreted by such cell types can be detected. In some aspects, the present invention provides a method that contains and detects locally derived biomarkers, which may be used for point-of-care diagnostic purposes (FIG. 1).

The sample that is analyzed by the devices and methods disclosed herein may be from any desirable source. For example, the sample may be collected from a wound exudate. In some embodiments, the sample may be obtained by aspirating the fluid from the aspirate of a syringe into the wound bed and transferring the contents into a collection vial. Alternatively, collection of wound exudates during “negative pressure wound therapy,” “reduced pressure therapy,” or “vacuum therapy” may be used for detection of biomarkers. In some embodiments, absorbent filter-paper may be placed within a separate device that titrates away a small amount of wound exudates during “negative pressure wound therapy,” “reduced pressure therapy,” or “vacuum therapy.” In other embodiments, the wound exudate may be collected directly on a wound dressing. In other embodiments, the sample may be collected from other sources. For example, the biomarkers may be extracted from tissue which is either adhered to dressing material (upon removal of the dressing from the wound) or from non-necrotic tissue removed during debridement of the wound. The extraction would possibly entail a soak (lysis) and/or a soak and grind step to release biomarkers.

The methods disclosed herein will indirectly and accurately measure cell presence by the secretion of cell-specific markers into the wound exudate rather than measuring cells directly, which can further damage the patient's tissue. Wound exudates often provide an accurate reflection of the extracellular environment of the wound and its relative health. This allows one to distinguish between the different phases of wound healing.

### B. BIOMARKERS

In some aspects, the devices and methods disclosed herein utilize biomarkers. A biomarker is a measurable biological substance, or signature, that serves as a specific indicator of normal or abnormal biologic processes, often useful for evaluation of pharmacologic responses to therapeutic interventions. Several biomarkers could form the basis for detection of neutrophils and fibroblasts in wounds, Detection of neutrophil-secreted factors and fibroblast-secreted factors may serve as specific biomarkers for the development of a ratio-based diagnostic for determining the phase of wound healing.

Any secreted biomarker may be used with the presently disclosed methods and devices. In some aspects, the biomarkers are neutrofil- or fibroblast-specific biomarkers. In other aspects, the biomarkers are secreted from M1 or M2 macrophages. Other cell-specific secreted biomarkers may be recognized by a person having ordinary skill in the art.

The secreted biomarkers may be used in any desirable combination with the disclosed methods and devices. For example, the methods and devices may use 1, 2, 3, 4, 5, 10, 15, 20, or more biomarkers. In some aspects, the method or device may use one or more neutrophil-secreted factors in combination with one or more fibroblast-secreted factors. In other embodiments, the method or device may use one or more factors secreted from M1 macrophages in combination with one or more factors secreted from M1 macrophages. Other desirable combinations of biomarkers may be recognized by a person having ordinary skill in the art.

1. Neutrophil-Secreted Factors

In some aspects, the devices and methods disclosed herein utilize biomarkers that are secreted from neutrophil cells, A person having ordinary skill in the art would recognize a variety of secreted neutrophil-specific biomarkers. In some embodiments, the neutrophil-secreted biomarker is myeloperoxidase (MPO), neutrophil elastase (nElastase), Human Neutrophil. Lipolin (HNL), Lactoferrin, Lysozyme, Neutrophil Gelatinase-Associated Lipocalin (NGAL), Human Neutrophil Elastase Anti-Neutrophil Cytoplasmic Antibodies (HNE ANCA's), MMP9, Proteinase 3, Serpin Peptidase Inhibitor Clade B and D, Reactive Oxygen Species (ROS), or Reactive Nitrogen Species (RNS).

2. Fibroblast-Secreted Factors

In some aspects, the devices and methods disclosed herein utilize biomarkers that are secreted from fibroblast cells. A person having ordinary skill in the art would recognize a variety of secreted fibroblast-specific biomarkers. In some embodiments, the fibroblast-secreted biomarker is FGF2, FGF10, Fibroblast-specific protein 1 (FSP1), prolyl-4-hydroxylase (5B5), Insulin Growth Factor-1 (IGF-1), Tetranectin, Collagen alpha 1, 2, and 3 chains, SERPINA1, or Compliment Components.

3. Other Biomarkers

In wound healing, M1 macrophages direct an inflammatory response while M2 macrophages initiate repair and angiogenesis. M2 macrophages, compared to M1 macrophages, produce low amount of tumor necrosis factor-α (TNF-α) and high amounts of other detectable markers. Therefore, distinction between M1 macrophages (that produce pro-inflammatory cytokines) and M2 macrophages (that act to dampen inflammatory responses and promote angiogenesis, tissue remodeling and repair) are also indicative of the phases of wound healing. Examples of useful biomarkers specific to M1 or M2 macrophages include, but are not limited to, calgranulin A/B, Cystatin A, S100 Calcium Binding Proteins (specific for M1) and CD163, CD204, CD206, AM-3K, CSF-1R (colony-stimulating factor-1 receptor), a specific marker of macrophages, EMR1 (epideimal growth factor module-containing mucin-like receptor 1), or F4/80 (specific for M2). In addition, an analysis of levels of pro-collagen or collagen versus fibronectin is also indicative of either the proliferative phase (collagen) or the inflammatory phase (fibronectin) of wound healing.

### C. RELATIONSHIP BETWEEN THE AMOUNTS OF THE BIOMARKERS

In some aspects, the disclosed methods and devices utilize the amount of a biomarker or the ratio between selected biomarkers to determine the phase of wound healing. In some embodiments, the ratio between a neutrophil-secreted factor and a fibroblast-secreted factor will be monitored. In such embodiments, an amount of the fibroblast-secreted factor that is greater than the amount of the neutrophil-secreted factor indicates that the wound from which the sample was collected is in the proliferation phase or is properly healing. In some embodiments, if the amount of the fibroblast-secreted factor is greater than the neutrophil-secreted factor by 2-fold or greater, then the wound is in the proliferative stage. In some embodiments, the ratio of neutrophil-secreted factor to fibroblast-secreted factor is 1:1.0, 1:1.1, 1:1.2, 1:1.3, 1:1.4, 1:1.5, 1:1.6, 1:1.7, 1:1.8, 1:1.9, 1:2.0, 1:3, 1:4, 1:5, 1:10, or greater or any ratio derivable in between. In contrast, an amount of the neutrophil-secreted factor that is greater than the amount of the fibroblast-secreted factor indicates that the wound from which the sample was collected is in the inflammation phase or is not properly healing. In some embodiments, if the amount of the neutrophil-secreted factor is greater than the fibroblast-secreted factor by 2-fold or greater, then the wound is in the inflammation stage. In other embodiments, the ratio of fibroblast-secreted factor to neutrophil-secreted factor is 1:1.0, 1:1.1, 1:1.2, 1:1.3, 1:1.4, 1:1.5, 1:1.6, 1:1.7, 1:1,8, 1:1.9, 1:2.0, 1:3, 1:4, 1:5, 1:10, or greater or any ratio derivable in between.

In some embodiments, the ratio between an M1-secreted factor and an M2-secreted factor will be monitored. In such embodiments, an amount of the M2-secreted factor that is greater than the amount of the M1-secreted factor indicates that the wound from which the sample was collected is in the proliferation phase or is properly healing. In some embodiments, if the amount of the M2-secreted factor is greater than the M1-secreted factor by 2-fold or greater, then the wound is in the proliferative stage. In some embodiments, the ratio of M1-secreted factor to M2-secreted factor is 1:1.0, 1:1.1, 1:1.2, 1:1.3, 1:1.4, 1:1.5, 1:1.6, 1:1.7, 1:1.8, 1:1.9, 1:2.0, 1:3, 1:4, 1:5, 1:10, or greater or any ratio derivable in between. In contrast, an amount of the M1-secreted factor that is greater than the amount of the M2-secreted factor indicates that the wound from which the sample was collected is in the inflammation phase or is not properly healing. In some embodiments, if the amount of the M1-secreted factor is greater than the M2-secreted factor by 2-fold or greater, then the wound is in the inflammation stage. In other embodiments, the ratio of M2-secreted factor to M1-secreted factor is 1:1.0, 1:1.1, 1:1.2, 1:1.3, 1:1.4, 1:1.5, 1:1.6, 1:1.7, 1:1.8, 1:1.9, 1:2.0, 1:3, 1:4, 1:5, 1:10, or greater or any ratio derivable in between.

Similar relationships could be applied to any combination of biomarkers where one or more biomarkers are secreted from a cell-type specific to the inflammation stage of wound healing and one or more biomarkers are secreted from a cell-type specific to the proliferation stage of wound healing.

### D. DEVICES

In some embodiments, the present invention provides devices that are useful to detect and/or visualize one or more biomarkers from wound fluid. These devices may comprise a surface and at least one agent that is specific to a desired biomarker. The surface may be any surface to which the desired agents may be attached, including but not limited to a wound dressing or a microarray.

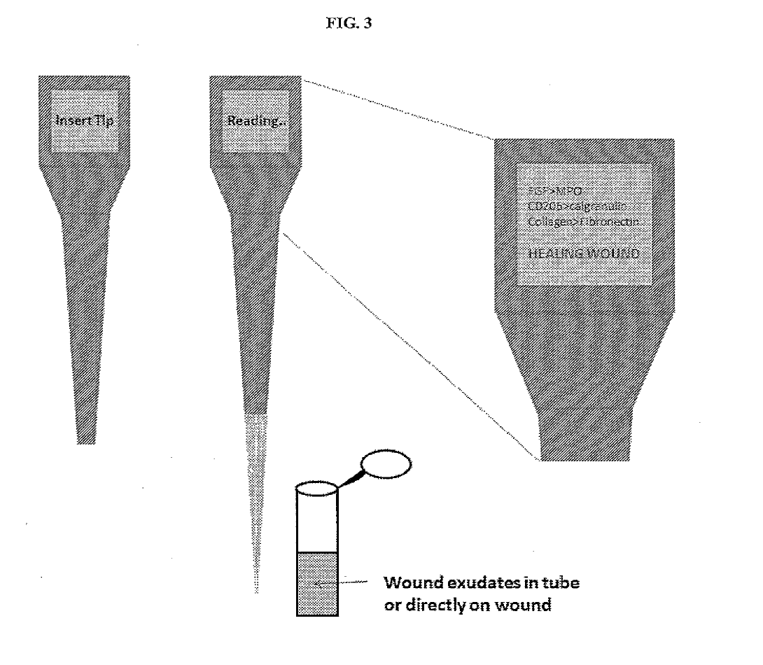

In some embodiments, the device may be a microfluidic device, e.g., similar to a pregnancy test. In other embodiments, the device may be in the tubing or tubing connectors of a device designed for “negative pressure wound therapy,” “reduced pressure therapy,” or “vacuum therapy.” In such a device, the tubing may change color when exudates with excessive neutrophil biomarkers or excessive fibroblast markers are present. In other embodiments, the device may be in the form of a canister or a sticker on the bottom of a canister that turns color. In still other embodiments, the device may be a handheld device that is programmed to detect the levels of these proteins and to digitally display a final reading (FIG. 3).

The agent specific to the biomarker may be any agent that can bind specifically to the desired biomarker. Examples include, but are not limited to, aptamers, ligands, antibodies, peptide sequences or other binding agents known to those having skill in the art.

Alternatively, collection of wound exudates during “negative pressure wound therapy,” “reduced pressure therapy,” or “vacuum therapy” may be used for detection of biomarkers. The absorbent filter-paper, covalently linked with aptamer, ligand, or antibody (FIG. 1), may be placed within a separate device that titrates away a small amount of wound exudates during “negative pressure wound therapy,” “reduced pressure therapy,” or “vacuum therapy.”

In some embodiments, the device is coated or bound with agents that bind to the biomarkers. In some embodiments, the agents may be bound chemically (ionic or covalently) or physically to the wound dressing. In some embodiments, the agents are bound by chemical grafting, polymer-binder, bi-functional chemical binding (e.g., silane or pluronic), hydrophilic or electrochemical binding, or plasma coating. In other embodiments, the device may be coated with the agents.

1. Wound Dressings

In some embodiments, the device may be a wound dressing. The wound dressing may contain one or more agents specific to a desired biomarker. The term “wound dressing” used herein is taken to include any pharmaceutically acceptable wound covering or support matrix. Examples of suitable wound dressing materials include, but are not limited to, a) films, including those of a semipermeable or a semi-occlusive nature such as polyurethane copolymers, polyurethane film, acrylamides, acrylates, paraffin, polysaccharides, cellophane and lanolin; h) hydrocolloids including carboxymethylcellulose protein constituents of gelatin, pectin, and complex polysaccharides including Acacia gum, guar gum and karaya, which may be utilized in the form of a flexible foam, formulated in polyurethane, or formulated as an adhesive mass such as polyisobutylene; c) polymers such as agar, starch or propylene glycol, which typically contain about 80% to about 90% water and are conventionally formulated as sheets, powders, pastes and gels in conjunction with cross-linked polymers such as polyethylene oxide, polyvinyl pyrollidone, acrylamide, propylene glycol; d) foams such as polysaccharide which consist of a hydrophilic open-celled contact surface and hydrophobic closed-cell polyurethane; e) impregnates including pine mesh gauze, paraffin and lanolin-coated gauze, polyethylene glycol-coated gauze, knitted viscose, rayon, and polyester; and f) cellulose-like polysaccharide such as alginates, including calcium alginate, which may be formulated as non-woven composites of fibers or spun into woven composites.

2. Microarrays

In some embodiments, the device may be a microarray. The microarray may have one or more agents specific to a desired biomarker disposed on its substrate. An “array” refers a device consisting of a substrate, typically a solid support having a surface adapted to receive and immobilize a plurality of different protein, peptide, and/or nucleic acid species (i.e., capture or detection reagents) that can used to determine the presence and/or amount of other molecules (i.e., analytes or biomarkers) in biological samples such as wound exudate. A “microarray” refers to an array wherein the different detection reagents are disposed on the substrate. In some embodiments, the agents may be bound chemically (ionic or covalently) or physically to the microarray.

### E. DETECTION METHODS

The detection of the biomarker presence may be performed in any manner known to those having skill in the art. In some embodiments, the method by which the biomarkers are detected may be related to the type of device used. In some embodiments, simultaneous detection of several biomarkers may be performed to provide a more accurate determination of the wound status.

In some embodiment, the biomarkers bind to the specific agent on the device. In such embodiments, the bound biomarkers may be detected by any appropriate method known to those having skill in the art. In some embodiments, the presence of the secreted biomarkers may be detected using ELISA, immunofluorescence test, microarray, luminescence test, blot, radioimmunoassay, Western blot or dot blot. In some embodiments, the presence of the secreted biomarkers may be detected using colorimetric detection methods. These methods may result in visual color changes that correlate with the presence or absence of wound healing biomarkers. In some embodiments, the presence of secreted biomarkers may be detected using Mass Spectrometry, Fourier transform infrared spectroscopy (FTIR), Polymerase Chain Reaction (PCR), Quantitative Real-Time PCR, or Northern Blot.

### F. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### HL60 and HDFa

The secreted levels of biomarkers of interest was assess in cultures of distinct cell types to allow one skilled in the art to assess biomarkers that are unique to each cell type and to determine levels of the unique biomarkers. The two cell types used were HL60 (Human Promyelocytic Leukemia) cells and HDFa (Adult Human Dermal Fibroblasts). The HL60 cells were induced to differentiate into neutrophil-like cells using 1.2% DMSO. Comparable numbers of cells were cultured for comparable amounts of time at 37° C. in 5% CO₂ Conditioned media (containing secreted factors from each cell type) was assessed for the presence of MPO, FGF2 and other analytes using a traditional ELISA technique (Table 1). ELISA, (Enzyme-linked immunosorbent assay), is a biochemical analysis tool used to detect the presence of an antigen (typically a protein) in a sample. Briefly, an unknown amount of antigen, in the test sample, is affixed specifically or non-specifically to a surface. A specific antibody is then applied over the surface so that it can bind to the test antigen. This antibody is typically linked to an enzyme which will be converted into a detectible signal (commonly a color change) upon addition of a chemical substrate.

TABLE 1

| | Negative | | |
|---|---|---|---|
| Analyte | Control | Positive Control | Test Samples |
| MPO | −−− | +++ | HL60+/HDFa−, HEKa− |
| Elastase | −−− | +++ | HL60+/HDFa−, HEKa− |
| Fibronectin | −−− | +++ | N/A |
| TNF-alpha-1 | −−− | +++ | N/A |
| MMP | −−− | +++ | N/A |
| FGF basic-2 | −−− | +++ | HL60+/HDFa+++ |
| TGF-Beta1 | −−− | +++ | HL60+/HDFa+ |

+++: within standard curve range;
−−−: below standard curve range
FGF: Fibroblast Growth Factor;
MPO: Myeloperoxidase;
MMP: Matrix Metalloproteinase;
TNF: Tumor Necrosis Factor;
TGF: Transforming Growth Factor;
HL60; Human Pro-myelocytic Leukemia Cell Line;
HDFa: Human Dermal Fibroblasts from an adult source;
HEKa: Human Epidermal Keratinocyte from an adult source;
N/A: not available

Table 2 shows the concentration range for the analyte that were measured in the discrete cell culture media during the experiment. For example, in HL-60 conditioned media, concentrations of MPO between 6.4-16 ng/mL were measured.

TABLE 2

| Analyte | Lowest concentration | | Highest concentration | |
|---|---|---|---|---|
| MPO | 6.4 | ng/ml | 16 | ng/ml |
| Elastase | 3125 | pg/ml | 6250 | pg/ml |
| FGF Basic | 10 | pg/ml | 20 | pg/ml |
| TFG-Beta1 | 125 | pg/ml | 250 | pg/ml |
| Fibronectin | 500 | ng/ml | 5 | μg/ml |
| TNF-alpha-1 | 200 | pg/ml | 6 | ng/ml |
| MMP | 200 | ng/ml | 13 | μg/ml |

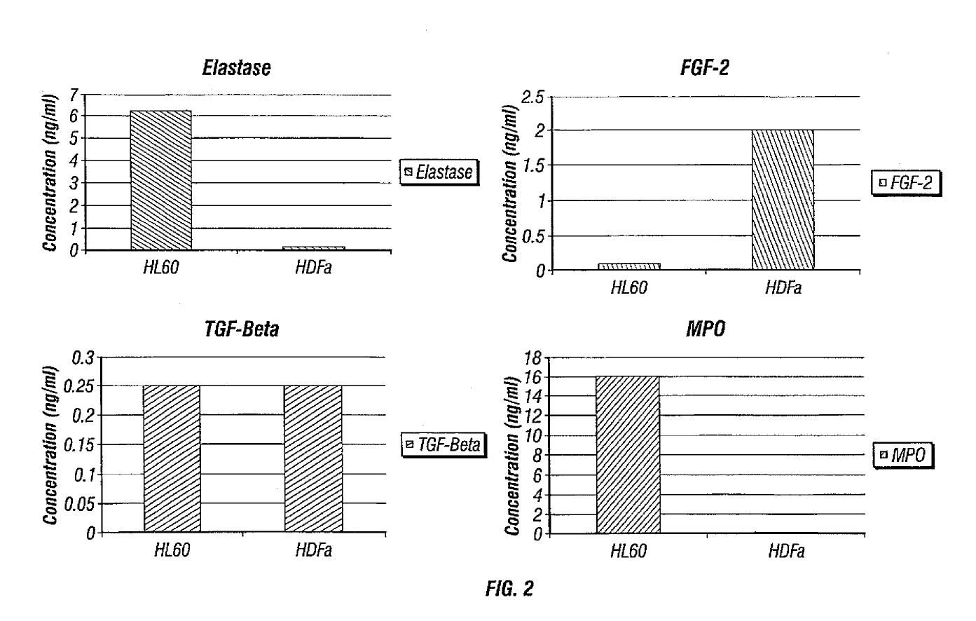

MPO was only detected in conditioned media taken form induced HL60-neutrophil cultures and was completely absent from conditioned media obtained from cultured fibroblasts. This demonstrates that MPO is a good candidate biomarker for the specific detection of neutrophils vs. fibroblasts (FIG. 2).

HL60 cells were also induced with 0.5% DMSO to attain a neutrophil-like culture. Induced HL60 and HDFa cells were then grown for comparable amounts of time using standard cell culture conditions. Conditioned media was tested for presence of NElastase, FGF-2, TGF-β1 and MPO.

FGF2 was only detected in conditioned media taken from fibroblasts and this biomarker was, in large part, absent from conditioned media obtained from cultured induced HL60 cells, signifying that this secreted biomarker is suitable for distinguishing fibroblasts from neutrophils in a wound environment. A control protein, TGF-β1, which is secreted by both neutrophils and fibroblasts, was included in this analysis and was found in conditioned media obtained from both cell types, as expected (FIG. 2).

### Example 2

### Quantification of MPO, nELASTASE, FGF2 and TGFβ from Human Wound Fluid

To compliment the analyses described above with the HL60 and HDF cell-line conditioned media, other analyses include the quantification of MPO, nElastase, FGF2 and TGFβ from human wound fluid obtained from various types of wounds provided by an outside vendor, Cureline, Inc. Specifically, wound exudative fluid (1 ml) is collected from consenting patients with the following types of wounds: diabetic foot ulcers, burns, pressure ulcers, venous stasis ulcers, and acute trauma which have no evidence of clinical infection. Inclusion criteria for these analyses include: male and female over 18 years of age with signed inform consent. Exclusion criteria include: active systemic infection, immunodeficiency disease, anemia (hematocrit<26), and/or receiving steroids, immunosuppressive, or cytotoxic medications. Test samples are collected first by recording the patients' demographic information (e.g., age of subject, gender, ethnicity, etc), and wound information including: duration of wound, date of future or previous surgeries, type of wound (acute or chronic), and previous wound treatment.

Test samples are prepared as follows: the wound area is traced and depth is measured in order to calculate the wound area. Based on the wound surface, the amount of saline to be added is calculated and recorded (e.g., for every 4 cm² of wound surface area, add 1 cc of saline). A semi-occlusive polyurethane film dressing (Tegaderm, 3M, St. Paul, Minn. or equivalent) is placed over the wound and wound fluid is collected by piercing the film dressing with a sterile 21 gauge stainless steel needle attached to a syringe and aspirating the contents into the wound bed between the polyurethane film dressing and wound bed itself prior to transfer into a cryo-supported polypropylene tube. Care is taken to avoid damage to underlying wound bed tissue, only the fluid is collected. The volume of wound fluid collected and time point is recorded and upon decanting the recovered wound fluid lavage into the cryo-supported polypropylene tube, the tubes are snap-frozen for storage and shipped on dry ice for analyses.

Analyses for MPO, nElastase, FGF2, and TGFβ is performed by ELISA as described above. The ratio between MPO:FGF2 and nElastase:FGF2 found within each wound fluid is determined and is used as diagnostic for the wound. A wound is properly healing if FGF2>MPO by 2-fold or greater or if FGF2>nElastase by 2-fold or greater. Levels or FGF2 lower than 2-fold relative to MPO or nElastase is indicative of a “blocked” or non-healing wound.

All of the compositions and/or methods disclosed and claimed herein can be made and executed without undue experimentation in light of the present disclosure. While the compositions and methods of this invention have been described in terms of some embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A wound diagnosis device comprising a first agent that is specific to a first biomarker and a second agent that is specific to a second biomarker, wherein the first biomarker is a neutrophil-secreted factor and the second biomarker is a fibroblast-secreted factor or the first and second biomarkers are secreted from M1 or M2 macrophages.

2-15. (canceled)

16. A method of determining the phase of wound healing comprising:
a) detecting the amount of a first biomarker and the amount of a second biomarker in a sample from a wound; and
b) comparing the amount of the first biomarker and the amount of the second biomarker,
wherein the first biomarker is a neutrophil-secreted factor and the second biomarker is a fibroblast-secreted factor or the first and second biomarkers are secreted from M1 or M2 macrophages and the relation between the amount of the first biomarker to the amount of the second biomarker indicates the phase of wound healing.

17. The method of claim 16, wherein the sample is wound fluid.

18. The method of claim 16, wherein the first biomarker has been secreted from neutrophil cells and the second biomarker has been secreted from fibroblast cells.

19. The method of claim 16, wherein if the amount of the first biomarker is greater than the amount of the second biomarker, the inflammatory phase of wound healing is indicated.

20. The method of claim 16, wherein if the amount of the first biomarker is less than the amount of the second biomarker, the proliferative phase of wound healing is indicated.

21. The method of claim 16, wherein the first biomarker is MPO and the second biomarker is FGF-2.

22. The method of claim 21, wherein if the amount of MPO is at least twice greater than the amount of FGF-2, then the inflammatory phase of wound healing is indicated.

23. The method of claim 21, wherein if the amount of MPO is half as much or less than the amount of FGF-2, then the proliferative stage of wound healing is indicated.

24. The method of claim 16, wherein the first biomarker is nElastase and the second biomarker is Fibroblast Growth Factor-10 (FGF-10).

25. The method of claim 16, wherein the first biomarker has been secreted from M1 macrophage cells and the second biomarker has been secreted from M2 macrophage cells.

26. The method of claim 25, wherein a larger amount of the first biomarker and a smaller amount of the second biomarker indicates the inflammatory phase of wound healing.

27. The method of claim 25, wherein a smaller amount of the first biomarker and a larger amount of the second biomarker indicates the proliferative phase of wound healing.

28. The method of claim 16, wherein the first biomarker is calgranulin A/B, TNF-α, MPO, or MMP and the second biomarker is CD204 or CD206.

29. The method of claim 28, wherein if the amount of CD204 or CD206 is less than twice the amount of calgranulin A/B, TNF-α, MPO, or MMP, then the wound is in the inflammatory phase of wound healing.

30. The method of claim 28, wherein if the amount of CD204 or CD206 is equal to or greater than twice the amount of calgranulin A/B, TNF-α, MPO, or MMP, then the wound is in the proliferative stage of wound healing.

31. The method of claim 16, wherein the first biomarker is pro-collagen or collagen and the second biomarker is fibronectin.

32. The method of claim 31, wherein if the amount of the first biomarker is greater than the amount of the second biomarker, the inflammatory phase of wound healing is indicated.

33. The method of claim 31, wherein if the amount of the first biomarker is less than the amount of the second biomarker, the proliferative phase of wound healing is indicated.

34.-49. (canceled)

50. A method of determining the phase of wound healing comprising:
a) detecting the amount of a first biomarker and the amount of a second biomarker in a sample from a wound; and
b) comparing the amount of the first biomarker and the amount of the second biomarker,
wherein the relative amount of the first biomarker to the second biomarker indicates the phase of wound healing.

51.-73. (canceled)
